SEQUENCE LISTING (30 sequences)

SEQ ID NO: 1 - protein, 23 aa, Artificial Sequence
  Synthetic
RKTAKRLGVYQSAINKLLLAAAK

SEQ ID NO: 2 - skipped
  Synthetic

SEQ ID NO: 3 - skipped
  Synthetic

SEQ ID NO: 4 - skipped
  Synthetic

SEQ ID NO: 5 - protein, 6 aa, Artificial Sequence
  Synthetic
VVVAAA

SEQ ID NO: 6 - protein, 6 aa, Artificial Sequence
  Synthetic
AAAVVV

SEQ ID NO: 7 - protein, 6 aa, Artificial Sequence
  Synthetic
LLLAAA

SEQ ID NO: 8 - protein, 6 aa, Artificial Sequence
  Synthetic
VVVVVV

SEQ ID NO: 9 - protein, 6 aa, Artificial Sequence
  Synthetic
VVVLLL

SEQ ID NO: 10 - protein, 6 aa, Artificial Sequence
  Synthetic
LLLVVV

SEQ ID NO: 11 - protein, 6 aa, Artificial Sequence
  Synthetic
AAAAAA

SEQ ID NO: 12 - protein, 7 aa, Artificial Sequence
  Synthetic
AAAAGGG

SEQ ID NO: 13 - protein, 6 aa, Artificial Sequence
  Synthetic
LLLLLL

SEQ ID NO: 14 - protein, 6 aa, Artificial Sequence
  Synthetic
AAAGGG

SEQ ID NO: 15 - protein, 6 aa, Artificial Sequence
  Synthetic
LLLGGG

SEQ ID NO: 16 - protein, 6 aa, Artificial Sequence
  Synthetic
AAALLL

SEQ ID NO: 17 - protein, 15 aa, Artificial Sequence
  Synthetic
RKTAKRLGVYQSAIN

SEQ ID NO: 18 - protein, 16 aa, Artificial Sequence
  Synthetic
RKTAKRLGVYQSAINK

SEQ ID NO: 19 - protein, 5 aa, Artificial Sequence
  Synthetic
PPRKV

SEQ ID NO: 20 - protein, 6 aa, Artificial Sequence
  Synthetic
PPRKVE

SEQ ID NO: 21 - protein, 17 aa, Artificial Sequence
  Synthetic
MRRAHHRRRRASHRRMR

SEQ ID NO: 22 - protein, 19 aa, Artificial Sequence
  Synthetic
MAPKRKSGVSKCETKCTPP

SEQ ID NO: 23 - protein, 29 aa, Artificial Sequence
  Synthetic
TSRANGSVGGEITKRLVRLAQQNMGGQFK

SEQ ID NO: 24 - protein, 30 aa, Artificial Sequence
  Synthetic
KDPAALKRARNTEAARRSRARKLQRMKQLE

SEQ ID NO: 25 - protein, 9 aa, Artificial Sequence
  Synthetic
FGRAXXXXX

SEQ ID NO: 26 - protein, 26 aa, Artificial Sequence
  Synthetic
DPAALKRARNTEAARRSRARKLQGGC

SEQ ID NO: 27 - protein, 52 aa, Artificial Sequence
  Synthetic
GRPRAINKHEQEQISRLLEKGHPPQQLAIIFGIGVSTLYRYFPASSIKKRMN

SEQ ID NO: 28 - protein, 43 aa, Artificial Sequence
  Synthetic
GRKRKIERDAVLNMWQQGLGASHISKTMNIARSTVYKVINESN

SEQ ID NO: 29 - protein, 7 aa, Artificial Sequence
  Synthetic
PKKKRKV

SEQ ID NO: 30 - protein, 15 aa, Artificial Sequence
  Synthetic
KRPAATKKAGQAKKK

(19) United States
(12) Patent Application Publication
(10) Pub. No.: US 2009/0098652 A1
(43) Pub. Date: Apr. 16, 2009

(54) SELF ASSEMBLING PEPTIDE SYSTEMS AND METHODS

(75) Inventors: Samuel I. Stupp, Chicago, IL (US); Chung-Yan Koh, Evanston, IL (US)

Correspondence Address: Casimir Jones, S.C., 440 Science Drive, Suite 203, Madison, WI, 53711, US

(73) Assignee: Northwestern University, Evanston, IL (US)

(21) Appl. No.: 12/193,541

(22) Filed: Aug. 18, 2008

Related U.S. Application Data
(60) Provisional application No. 60/956,588, filed on Aug. 17, 2007.

(51) Int. Cl. C12N 15/87; C07K 7/00

(52) U.S. Cl. 435/455; 530/330; 530/329; 530/326

(57) ABSTRACT

The present invention provides a self-assembling peptide system which utilizes a bioactive sequence which enhances transfection efficiency. In particular, the present invention provides compositions and methods for transfecting aggregates of cells at a higher efficiency.

### CROSS-REFERENCE TO RELATED APPLICATION

The present application claims priority to U.S. Provisional Patent Application Ser. No. 60/956,588, filed Aug. 17, 2007, the entire disclosure of which is herein incorporated by reference in its entirety.

### STATEMENT REGARDING FEDERALLY SPONSORED RESEARCH OR DEVELOPMENT

This invention was made with government support under grant no. 5R01EB003806 awarded by the National Institute of Biomedical Imaging and BioEngineering. The government has certain rights in the invention.

### FIELD OF THE INVENTION

The present invention provides a self-assembling peptide system which utilizes a bioactive sequence which enhances transfection efficiency. In particular, the present invention provides compositions and methods for transfecting aggregates of cells at a higher efficiency.

### BACKGROUND OF THE INVENTION

Transfection is the process by which foreign DNA is introduced to a cell, is transported from the cell membrane to the nucleus, and is then put through the cellular machinery in order to express the proteins for which it encodes. There are a variety of transfection systems available on the market; mechanical, electrical, viral, and chemical methods have been developed. The mechanical methods (direct injection, gene gun) are typically very hard on the cells, as it physically compromises the cell and nuclear membranes in order to deliver the DNA to the target. Electrical methods (electroporation, nucleofection) are thought to work by forcing open pores in the cell by electrical current. For several lines, this method has proven to be fatal to the cells. Viral methods are highly efficient and effective methods of transfection, but carry a large risk of incorporating viral DNA in the host cell as well as random insertion of the DNA into the host genome. This has been proven to increase the rate of formation of genetic abnormalities and increases the likelihood of cancerous development in the virally infected cells. As such, what is needed are transfection methods that are gentle on cells and effective.

### SUMMARY OF THE INVENTION

The present invention provides a self-assembling peptide system which utilizes a bioactive sequence which enhances transfection efficiency. In particular, the present invention provides compositions and methods for transfecting aggregates of cells at a higher efficiency. In some embodiments, the present invention provides a composition comprising one or more peptide amphiphiles, comprising a non-peptidic hydrophobic component covalently linked to structural peptide segment and a hydrophilic, DNA-binding peptide segment. In some embodiments, the structural peptide segment is configured to form a β-sheet. In some embodiments, the structural peptide segment is selected from any of, but not limited to SEQ ID NO. 5-16. In some embodiments, the DNA-binding peptide comprises RKTAKRLGVYQSAIN (SEQ ID NO 17). In some embodiments, the hydrophobic component is a single, linear alkyl chain of the formula: CₙH₂ₙ₋₁O—, where n=6−22. In some embodiments, the structural peptide segment is LLLAAA. In some embodiments, the hydrophobic component is a palmitoyl moiety (C₁₆H₃₁O—).

In some embodiments, the present invention provides a composition comprising one or more peptide amphiphiles, comprising a non-peptidic hydrophobic component covalently linked to structural peptide segment and a hydrophilic, nuclear localization signaling segment. In some embodiments, the structural peptide segment is configured to form a β-sheet. In some embodiments, the structural peptide segment is selected from any of, but not limited to SEQ ID NO. 5-16. In some embodiments, the nuclear localization signal is a peptide comprising PPRKV (SEQ ID NO 19). In some embodiments, the hydrophobic component is a single, linear alkyl chain of the formula: CₙH₂ₙ₋₁O—, where n=6−22. In some embodiments, the structural peptide segment is LLLAAA. In some embodiments, the hydrophobic component is a palmitoyl moiety (C₁₆H₃₁O—).

In some embodiments, the present invention provides a composition comprising one or more peptide amphiphiles, comprising a non-peptidic hydrophobic component covalently linked to structural peptide segment and a hydrophilic, DNA-binding peptide segment; and further comprises another peptide amphiphile comprising a non-peptidic hydrophobic component covalently linked to a structural peptide segment and a hydrophilic nuclear localization signaling segment. In some embodiments, the structural peptide segment is configured to form a β-sheet. In some embodiments, the structural peptide segment is selected from any of, but not limited to SEQ ID NO. 5-16. In some embodiments, the nuclear localization signal is a peptide comprising PPRKV (SEQ ID NO 19). In some embodiments, the hydrophobic component is a single, linear alkyl chain of the formula: CₙH₂ₙ₋₁O—, where n=6−22.

In some embodiments, the present invention provides a kit comprising one or more peptide amphiphiles, described above, or elsewhere herein.

In some embodiments, the present invention provides a method of transfecting cells, comprising: a) providing a plurality of cells, a composition comprising one more more of the peptide amphiphiles described herein; b) contacting the composition with the DNA target; and subsequently, contacting the composition with the plurality of cells. In some embodiments, the plurality of cells are a cellular aggregate. In some embodiments, the cellular aggregate is selected from the group consisting of stem cells or organoids. In some embodiments, the organoids comprise Islets of Langerhans.

### DESCRIPTION OF THE FIGURES

FIG. 1a shows the chemical structure of one possible nuclear localization signal peptide amphiphile. FIG. 1b shows the chemical structure of one possible DNA-binding peptide amphiphile.

FIG. 2 shows transfection of P19 cells in suspension aggregates is greatly enhanced by the PA system compared to the Lipofectamine system. Peptides without the beta-sheet forming region are shown to have lower transfection efficiency than when in the nanofiber structure. In the figure, “peptide amphiphile” is the DNA-binding PA with sequence RKTAKRLGVYQSAINKLLLAAAK(N^{ε}-palmitoyl) and “peptide” is a peptide with sequence RKTAKRLGVYQSAINK.

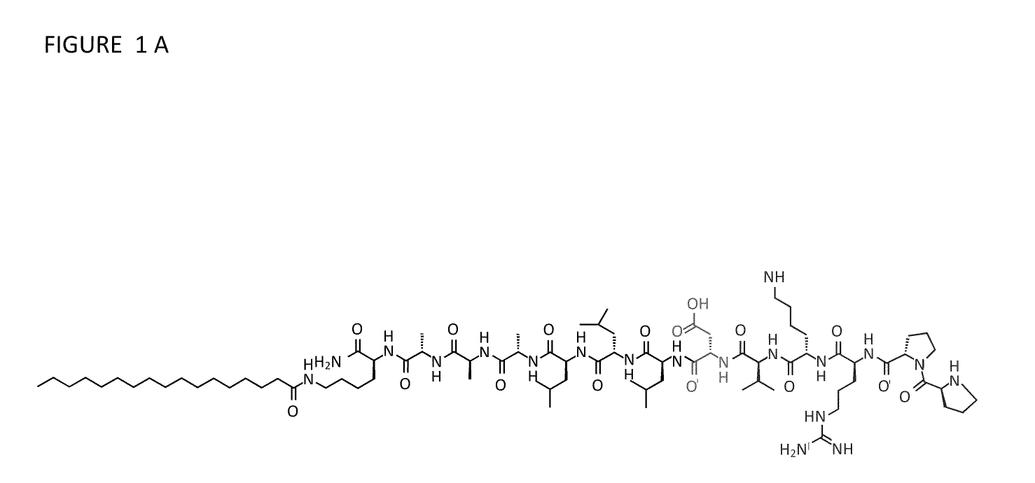

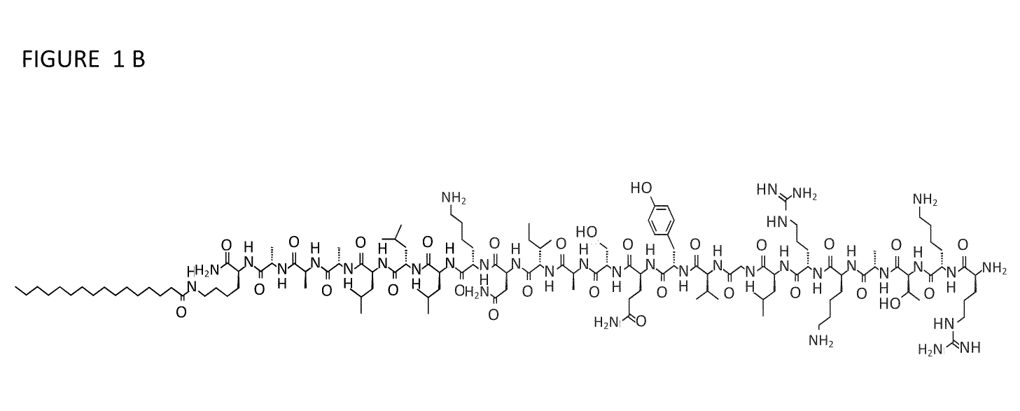

### DEFINITIONS

The terms “protein” and “polypeptide” refer to compounds comprising amino acids joined via peptide bonds and are used interchangeably.

As used herein, where “amino acid sequence” is recited herein to refer to an amino acid sequence of a protein or peptide molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The term “a polynucleotide having a nucleotide sequence encoding a gene” or “a polynucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “vector” refers to nucleic acid molecules that transfer DNA segment(s) from one cell to another. The term “vehicle” is sometimes used interchangeably with “vector.”

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “type of nucleic acid” refers to a characteristic or property of a nucleic acid that can distinguish it from another nucleic acid, such as a difference in sequence or in physical form, such as occurs in different expression vectors, or as occurs with the presence of DNA and RNA, or as occurs with the presence of linear and super-coiled DNA, or as occurs with the presence of coding regions which encode different proteins, or as occurs with the presence of different control elements, or control elements which differ amongst themselves.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene which confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat Nos., 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, .beta.-galactosidase, alkaline phosphatase, and horse radish peroxidase.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNA s which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

As used herein, the term “nuclear localization signal” means a molecule such as an amino acid sequence known to, in vivo or in culture, direct a molecule disposed in the cytoplasm of a cell across the nuclear membrane and into the nucleus of the cell. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 50, 25, 15, 12, 10, 8, 7, 6, 5 or 4 amino acids, but will preferably comprise an amino acid sequence known to function as a nuclear localization signal (NLS).

As used herein, a “cationic” lipid is one having a positive ionic character. Exemplary cationic lipids include dimethyldioctadecylammonium (DDAB), 1,2-diolelyloxy-3-(trimethylamino)propane (DOTAP), N-[1-(2,3,-dioleyloxy)propyl]-N,N-dimethyl-N-hydroxyethylammonium bromide (DORIE), N-[1-(2,3-dioleyloxy)propyl]-N,N,N-trimethylammonium chloride (DOTMA), dioleoylphosphatidylethanolamine (DOPE), and 3.beta.[N-(N′,N′-dimethylaminoethane)carbamoyl] cholesterol (DC-Chol).

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on). Thus, nucleotide sequences of the present invention can be engineered in order to introduce or alter a CBF3 coding sequence for a variety of reasons, including but not limited to initiating the production of environmental stress tolerance; alterations that modify the cloning, processing and/or expression of the gene product (such alterations include inserting new restriction sites and changing codon preference), as well as varying the protein function activity (such changes include but are not limited to differing binding kinetics to nucleic acid and/or protein or protein complexes or nucleic acid/protein complexes, differing binding inhibitor affinities or effectiveness, differing reaction kinetics, varying subcellular localization, and varying protein processing and/or stability).

As used herein, the terms “nanofiber” and “high aspect ratio nanostructure” refer interchangeably to an elongated or threadlike filament having a diameter of less than 100 nanometers. “High aspect ratio” refers to a ratio of length-to-diameter of greater than 10:1.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties of those components.

As used herein, the term “scaffold” and refers to a natural or synthetic structure or meshwork of structures with open porosity that is extended in space and provides mechanical or other support for the growth of living tissue, either in the body or in vitro.

As used herein, the term “peptide amphiphile” and the abbreviation “PA” refer to a molecule that, at a minimum, includes a non-peptide hydrophobic segment covalently linked to a structural peptide segment and a hydrophilic peptide segment. The peptide amphiphile may express a net charge at physiological pH, either a net positive or negative net charge, or may be zwitterionic (i.e., carrying both positive and negative charges).

As used herein, the term “hydrophobic moiety” refers to the hydrocarbon chain disposed at one terminus of a peptide amphiphile. This moiety may be herein and elsewhere referred to as the “hydrophobic component” or “hydrophobic segment”. The hydrophilic segment should be of a sufficient length to provide amphiphilic behavior and micelle formation in water or another polar solvent system.

As used herein, the term “structural peptide segment” refers to the intermediate amino acid sequence of the peptide amphiphile molecule generally composed of three to ten amino acid residues with non-polar, uncharged side chains, selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (O), Leu (L), Thr (T), Ala (A), Gly (G), (listed in order of their propensity to form beta sheets). However, non-naturally occurring amino acids of similar beta-sheet forming propensity may also be used. In a preferred embodiment, a strong and a weak beta sheet former are used in combination, for example taking the form (X_{A})_{Na}(X_{B})_{Nb}, where X_{A} and X_{B} are selected from A, L, V and G and Na and Nb are 2, 3 or 4. Illustrative examples include (SEQ ID NOs: 5-16). In a more preferred embodiment, the structural peptide segment is LLLAAA (SEQ ID NO. 7) or one of the following structural peptide segments including the following: VVVAAA (SEQ ID NO. 5), AAAVVV (SEQ ID NO. 6), VVVVVV (SEQ ID NO. 8), VVVLLL (SEQ ID NO. 9), LLLVVV (SEQ ID NO. 10), AAAAAA (SEQ ID NO. 11), AAAAGGG (SEQ ID NO. 12), LLLLLL (SEQ ID NO. 13), AAAGGG (SEQ ID NO. 14), LLLGGG (SEQ ID NO. 15), or AAALLL (SEQ ID NO. 16).

As used herein the term “bioactive agent” refers to substances which are capable of exerting a detectable biological effect in vitro and/or in vivo.

### DETAILED DESCRIPTION OF THE INVENTION

The present invention provides a self-assembling peptide system which utilizes a peptide sequence which enhances transfection efficiency.

In some embodiments, the compositions and methods of the present invention find use in the transfection of aggregates of cells with higher efficiency and more completely than current reagents on the market.

LIPOFECTAMINE is a product from Invitrogen Inc. which is the industry standard for lipofection. It is a cationic lipid which complexes with DNA in order to decrease the effective charge of the complex, thus increasing the likelihood that the complex will pass through the cell membrane. The reagent can be seen as a passive carrier which does not attempt to direct the fate of the complex, it merely allows transfection to be a possibility. With established protocols, it is typically not very toxic to the cells and has good transfection efficiency of cells on a surface in a monolayer.

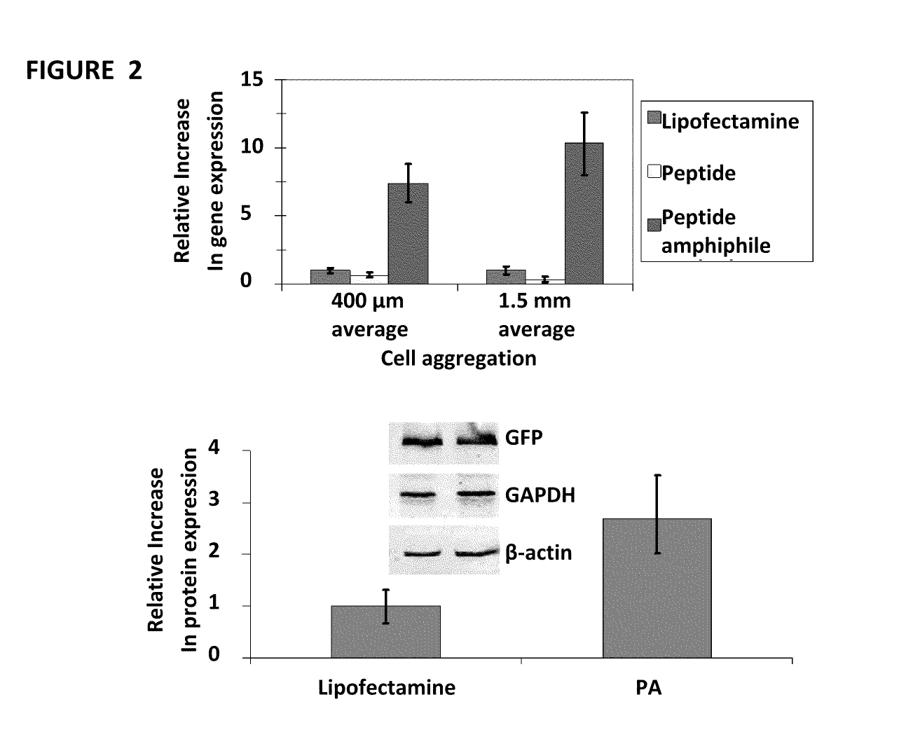

In some embodiments, the invention described here improves on this system by incorporating one or more peptides which are bioactive and direct the complex to the nucleus. It is designed, in some embodiments, to form an alpha helix which binds to the major groove of the double helix of DNA. In addition, it is a self-assembling peptide amphiphile (PA) which forms high aspect ratio nanostructures. When complexed with DNA, these nanostructures are, on average, smaller than the nanostructures produced by LIPOFECAMINE as determined by dynamic light scattering. The present invention is not limited to a particular mechanism. Indeed, an understanding of the mechanism is not necessary to practice the present invention. Nonetheless, it is contemplated that the system works by complexing and condensing the DNA through specific binding to the major groove and electrostatic screening. This DNA-PA complex is similar to the LIPOFECTAMINE complex, however it includes a peptide region containing a nuclear localization signal which directs the cell to move this complex through the cytoplasm and into the nucleus where the genes encoded in the DNA are subsequently expressed. When the peptide amphiphile system is placed in culture with a cellular aggregate, it self-assembles into a network of nanofibers which creates an internal scaffold. This scaffold penetrates to the core of the cellular aggregates, thus providing an increased effect. The improvements of this system over the LIPOFECTAMINE system are particularly useful in cellular aggregates in which a large increase in transfection is seen both in the number of mRNA transcripts contained within the cells (qPCR) and in the actual protein production of GFP (Western blot). Up to 10-fold increase in the mRNA transcript and threefold increase in the protein expression has been seen compared to LIPOFECTAMINE (see FIG. 2).

In gene delivery using non-viral vectors, two important factors are cytotoxicity and efficiency. While viral vectors may be highly efficient, the immunogenicity of the virus itself is often detrimental. In experiments conducted during development of embodiments of the present invention, a purely synthetic, self-assembling lipofection system is able to efficiency transfect large aggregates of cells while maintaining low cytotoxicity. Commercially available standard transfection reagents such as lipofectamine are highly efficient transfection agents for monolayers of cells, but, for suspension aggregates that efficiency is commonly known to decrease dramatically.

In experiments conducted during development of embodiments of the present invention, transmission electron microscopy (TEM) indicated that the morphology of the PA system changed dramatically upon addition of circular, plasmid DNA. Coupled with the electrophoretic mobility shift assay (EMSA) and DNA footprinting, experiments conducted during development of embodiments of the present invention point to an interaction of the DNA and the PA, although the present invention is not limited to any particular mechanism of action and an understanding of the mechanism of action is not necessary to practice the present invention. As all detectable DNA was shifted by the PA, it would seem that the interaction is strong enough to bind the DNA under electric fields in buffered systems.

Experiments conducted during the development of embodiments of the present invention, with a histidine hexamer incorporated in the DNA binding PA show that in P19 culture, a PA system is able to persist through two weeks of culture in a bioactive form. The histidine hexamer was chosen as it is not a peptide sequence that the embryonic stem cells should be able to express on their own.

Physical concentration of DNA at the surface of cells has been shown to increase transfection efficiency. Since the His₆ PA can be seen surrounding cells in the aggregate, this could be an explanation for the increased performance of the DNA-binding PA system, although the present invention is not limited to any particular mechanism of action and an understanding of the mechanism of action is not necessary to practice the present invention. The presence of the nuclear localization signal may also account for increase in transfection efficiency. The peptide control showed poor results compared to both the lipofectamine system and the PA system, suggesting that the self-assembled nanostructure is essential for the effect and also supporting the hypothesis of physical concentration of DNA at the cellular surface by the nanostructures. As the peptide without the β-sheet forming region is unable to self-assemble into nanofibers, there is no internal scaffold for the cells which brings the DNA to the cell surface.

### I. Compositions

In some embodiments, the materials of the present invention comprise one or more types of segments, a DNA binding portion and a nuclear localization portion. In some embodiments, one or both of the DNA binding portion and the nuclear localization portion are amphipathic. In some embodiments the DNA binding portion has a peptide moiety capable of binding to DNA, and a hydrophobic moiety. In some embodiments the nuclear localization portion has a peptide moiety, capable of directing transport to the nucleus, and a hydrophobic moiety. Thus, in embodiments, a composition is provided that is a mixture of a first composition comprising a peptide amphiphile having nuclear localization function and a second composition comprising a peptide amphiphile having a nucleic acid binding function.

In some embodiments of the present invention, the DNA binding PA and/or the nuclear localization PA are configured to self-assemble into nanostructures. In some embodiments, the present invention forms nanostructures when mixed with DNA. In some embodiments, hydrophobic interactions between the hydrophobic ends of the DNA binding portion and the nuclear localization portion bring the two portions together, although the present invention is not limited to any particular mechanism of action and an understanding of the mechanism of action is not necessary to practice the present invention. In some embodiments, hydrophobic interactions between the DNA binding portion and the nuclear localization portion result in self-assembly of the peptide amphiphiles into a higher order structure. In some embodiments, the self-assembly of the DNA binding portion and the nuclear localization portion in a higher order structure results in a complex in which the nuclear localization portion can direct the DNA binding portion to the nucleus.

In some embodiments, the present invention comprises a composition in which a DNA binding moiety and a nuclear localization moiety are provided in a single molecule. In some embodiments, a composition is provided having both DNA binding functionality and nuclear localization functionality. In some embodiments the present invention provides a peptide amphiphile of the sequence RKTAKRLGVYQSAINKK(N^{ε}-palmitoyl)-PPRKV)LLLAAAK(N^{ε}-palmitoyl) (SEQ ID NO. 17). In some embodiments, the DNA binding moiety and nuclear localization moiety are covalently bonded. In some embodiments, the present invention has a branched configuration (e.g. containing one or more of each of the nuclear localization and DNA binding moieties connected to one another or a scaffold).

In some embodiments, the present invention comprises a DNA binding portion. In some embodiments, the DNA binding portion comprises any of the DNA binding peptides described below (section a), covalently linked to any of the hydrophobic moieties described below (section c) and a structural moiety (section e). In some embodiments, the present invention comprises a nuclear localization portion. In some embodiments, the nuclear localization portion comprises any of the nuclear localization moieties described below (section b), covalently linked to any of the hydrophobic moieties described below (section c) and a structural moiety (section e).

a. DNA Binding Portion.

In some embodiments, the DNA binding portion is an amphiphilic composition composed of a DNA binding moiety and a hydrophobic moiety. The DNA binding moiety is a molecule, composition, compound, or complex configured to bind to DNA. In a preferred embodiment, the DNA binding moiety is a DNA binding peptide. The DNA binding peptide is preferably located in a peptide amphiphile between the hydrophobic moiety and a structural moiety. In some embodiments, the DNA binding peptide is a bioactive peptide capable of binding DNA. In some embodiments, the DNA binding peptide comprises a sequence, secondary fold, or tertiary fold which allows it to bind to DNA. In some embodiments, the DNA binding peptide is folded into a motif selected from, but not limited to: helix-turn-helix, zinc finger, leucine zipper, winged helix, winged helix-turn-helix, helix-loop-helix, immunoglobulin fold, and B3 domain. In some embodiments, the DNA binding peptide is a fragment of a known DNA binding protein (e.g. transcription factors, nucleases, histones, etc). In some exemplary embodiments, the peptide sequence of the DNA-binding peptide of the present invention is RKTAKRLGVYQSAINKLLLAAAK (SEQ ID NO. 1), or mutants and variants thereof. In other preferred embodiments, the DNA-binding peptide RKTAKRLGVYQSAIN (SEQ ID NO. 17) or RKTAKRLGVYQSAINK (SEQ ID NO. 18) is covalently attached to other structural peptides selected from any of SEQ ID NO. 5-16. In some embodiments, the peptide sequence of the DNA binding moiety may be any length amino acid chain capable of binding DNA (e.g. 5 amino acids, 10 amino acids, 15 amino acids, 20 amino acids, 30, amino acids, 40 amino acids, 50 amino acids, 100 amino acids, etc.). Additional DNA binding signals are known in the art and may be utilized. These DNA binding signals often form α-helices and have amino acid residues which are positively charged at pH 7.4. Some other peptide sequences which have may have similar properties are therefore rich in lysine, arginine, alanine, glycine, histidine, and leucine. Additional DNA binding sequences include, but are not limited to, MRRAHHRRRRASHRRMR (SEQ ID NO:21), MAPKRKSGVSKCETKCTPP (SEQ ID NO:22), TSRANGSVGGEITKRLVRLAQQNMGGQFK (SEQ ID NO:23), KDPAALKRARNTEAARRSRARKLQRMKQLE (SEQ ID NO:24), FGRAXXXXX- where X is any amino acid (SEQ ID NO:25), DPAALKRARNTEAARRSRARKLQGGC (SEQ ID NO:26), GRPRAINKHEQEQISRLLEKGHPPQQLAIIFGIGVSTLYRYFPASSIKKRMN (SEQ ID NO:27), and GRKRKIERDAVLNMWQQGLGASHISKTMNIARSTVYKVINESN (SEQ ID NO:28).

b. Nuclear Localization Portion.

In some embodiments, the nuclear localization portion is an amphiphilic composition composed of a nuclear localization moiety and a hydrophobic moiety. The nuclear localization moiety is a molecule, composition, compound, or complex configured to be delivered to the cell nucleus. In some preferred embodiments, the nuclear localization moiety is a nuclear localization peptide. The nuclear localization peptide is preferably located in a peptide amphiphile between a hydrophobic moiety and a structural moiety. The nuclear localization peptide is a bioactive nuclear localization signal peptide capable of directing itself to the cell nucleus. In some embodiments the nuclear localization moiety is capable of directing compositions to which it is attached covalently, non-covalently or through a series of interactions, to the cell nucleus. In some embodiments the nuclear localization moiety directs itself and any attached cargo through the nuclear envelope or nuclear pore complex. In some embodiments, secondary factors, such as transport proteins are involved in directing the nuclear localization moiety, and any attached cargo, to the cell nucleus (e.g. importin). In some embodiments, the nuclear localization peptide comprises a sequence, secondary fold, or tertiary fold, which targets the peptide and any attached cargo to the cell nucleus. In some exemplary embodiments, the peptide sequence of the nuclear localization signal is PPRKVELLLAAAK, or mutants and variants thereof. In other preferred embodiments, the nuclear localization signal PPRKV (SEQ ID NO. 19) or PPRKVE (SEQ ID NO. 20) is covalently attached to other structural peptides selected from any of SEQ ID NO. 5-16. In some embodiments, the nuclear localization signal is any peptide which fits the general sequence PₘBₙXₒ (m is the number of prolines (P), where m is between 1 and 3; n is the number of basic amino acids (B), such as histidine, lysine, ornithine, and/or arginine and n is between 2 and 10; o is the number of nonpolar amino acids such as alanine, glycine, leucine, isoleucine, and/or valine, and o is between 1 and 3). In some embodiments, the peptide sequence of the nuclear localization signal may be any length amino acid chain capable of targeting the peptide to the cell nucleus (e.g. 5 amino acids, 10 amino acids, 15 amino acids, 20 amino acids, 30, amino acids, 40 amino acids, 50 amino acids, 100 amino acids, etc.). Additional nuclear localization signals may be utilized, including, but not limited to PKKKRKV (SEQ ID NO:29) and KRPAATKKAGQAKKK (SEQ ID NO:30).

c. Hydrophobic Moiety.

In some embodiments, one or more the DNA binding portion and the nuclear localization portion of the present invention contain a hydrophobic moiety. In some embodiments, the hydrophobic moiety is covalently attached to a DNA binding moiety (e.g. DNA binding peptide) to form the DNA binding portion of the present invention. In some embodiments, the hydrophobic moiety is covalently attached to a nuclear localization moiety (e.g. nuclear localization peptide) to form the nuclear localization portion of the present invention. In some embodiments, hydrophobic interactions between the hydrophobic moieties of the present invention are configured to result in self-assembly of the DNA binding portion and nuclear localization portion into a higher order structure, although the present invention is not limited to any particular mechanism of action and an understanding of the mechanism of action is not necessary to practice the present invention.

In some embodiments, the hydrophobic moiety is any non-peptidic synthetic or naturally occurring molecule that exhibits hydrophobicity (e.g. alkanes, oils, fats, greasy substances, etc). In some embodiments the hydrophobic moiety is any lipid (e.g. fatty acyls, glycerolipids, glycerophospholipids, sphingolipids, sterol lipids, prenol lipids, saccharolipids, and polyketides). In some embodiments, the hydrophobic moiety is any fatty acid (e.g. palmitic acid, caprylic acid, myristic acid, stearic acid, arachidic acid, behenic acid, etc). In some embodiments, the hydrophobic moiety is a saturated fatty acid, an unsaturated fatty acid, a monounsaturated fatty acid, or a polyunsaturated fatty acid. In some embodiments the hydrophobic moiety is a fatty acid comprising a chain of 6 to 80 carbon atoms (preferably 12 to 24 carbon atoms). In some embodiments the hydrophobic moiety is a straight-chain fatty acid, a branched-chain fatty acid, or a fatty acid containing a functional group (e.g. acetylenic bonds, epoxy-, hydroxy- or keto groups, ring structures (e.g. cyclopropane, cyclopropene, cyclopentene, furan, cyclohexyl, etc.), or a coenzyme A moiety (e.g. acyl CoA (e.g. palmitoyl-CoA))).

In some embodiments, the hydrophobic moiety is attached to either the DNA binding moiety or the nuclear localization moiety at a lysine residue at the C-terminus of the DNA binding moiety or the nuclear localization moiety. In some embodiments, attachment of the hydrophobic moiety to the DNA binding moiety or the nuclear localization moiety can occur at the N-terminus, or at other amino acids. In the context of the present invention, the hydrophobic segment preferably comprises a single, linear alkyl chain of the formula: CₙH₂ₙ₋₁O—, where n=6−22. A particularly preferred hydrophobic is palmitic acid (C₁₆H₃₁O—).

d. Cationic Lipid

In some embodiments, the present invention further comprises a cationic lipid (e.g. DOTAP, SAINT-2, DC-Chol, GS1, etc.). The cationic included in the present invention is generally a vesicle-forming lipid. In a preferred embodiment, the present invention comprises between about 20-80 mole percent cationic lipids. The cationic vesicle-forming lipid is one whose polar head group with a net positive charge, at the operational pH (e.g., pH 4-9). Typical examples include phospholipids, such as phosphatidylethanolamine, whose polar head groups are derivatized with a positive moiety (e.g., lysine, as illustrated, for example, for the lipid DOPE derivatized with L-lysine (LYS-DOPE) (Guo, et al., 1993). Also included in this class are the glycolipids, such as cerebrosides and gangliosides having a cationic polar head-group.

Another cationic vesicle-forming lipid which may be employed is cholesterol amine and related cationic sterols. Exemplary cationic lipids include 1,2-diolelyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3.beta.[N-(N′,N′-dimethylaminoethane)carbamoly]cholesterol (DC-Chol); and dimethyldioctadecylammonium (DDAB). In a preferred embodiment, the cationic lipid is 1,2-diolelyloxy-3-(trimethylamino)propane (DOTAP).

In another embodiment, the cationic lipid may be a neutral cationic lipid, that is, a lipid that at physiologic pH of 7.4 is predominantly (e.g., greater than 50%, neutral in charge but at a selected pH value less than physiologic pH tends to have a positive charge).

e. Structural Moiety

As described above, a structural moiety or “structural peptide segment” may be present in a peptide amphiphile of the present invention. It is preferably an intermediate amino acid sequence of the peptide amphiphile molecule generally composed of three to ten amino acid residues with non-polar, uncharged side chains, selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (O), Leu (L), Thr (T), Ala (A), Gly (G), (listed in order of their propensity to form beta sheets). However, non-naturally occurring amino acids of similar beta-sheet forming propensity may also be used. In a preferred embodiment, a strong and a weak beta sheet former are used in combination, for example taking the form (X_{A})_{Na}(X_{B})_{Nb}, where X_{A} and X_{B} are selected from A, L, V and G and Na and Nb are 2, 3 or 4. Illustrative examples include (SEQ ID NOs: 5-16). In a more preferred embodiment, the structural peptide segment is LLLAAA (SEQ ID NO. 7) or one of the following structural peptide segment including the following:VVVAAA, AAAVVV, VVVVVV, VVVLLL, LLLVVV, AAAAAA, AAAAGGG, LLLLLL, AAAGGG, LLLGGG, and AAALLL.

### II. Methods

The systems and methods of the present invention find use in the transfection of any number of cell types. Cells may be in vitro, in culture, ex vivo, or in vivo.

In some embodiments, the systems and methods of the present invention find use in research applications. In some embodiments, the systems are sold as kits for transfection.

In some embodiments, the systems and methods of the present invention find use in the nonviral transfection of cellular aggregates in suspension. For examples, Islets of Langerhans are large aggregates of cells which benefit from transfection of anti-apoptotic factors. The maximum transfection efficiency seen in these systems thus far in literature has been 36% (Lakey et al., Cell Transplantation, Volume 10, Number 8, 2001, pp. 697-708(12)). The systems and methods of the present invention improve upon the known methods for transfection of cellular aggregates.

The compounds of the present invention may also be conjugated to or mixed with or used in conjunction with a variety of useful molecules and substances such as proteins, peptides, growth factors and the like to enhance cell-targeting, uptake, internalization, nuclear targeting and expression.

The present invention also includes self-assembling peptide amphiphiles (PA) comprising one or more compounds of the present invention or mixtures thereof. Self-assembling PA may be combined with one or more components and/or transfection enhancers.

The transfection methods of the present invention, employing the compounds or compositions (such as those described above) of the present invention or mixtures thereof, can be applied to in vitro and in vivo transfection of cells, particularly to transfection of eukaryotic cells or tissues including animal cells, human cells, insect cells, plant cells, avian cells, fish cells, mammalian cells and the like.

The present invention is not limited to the introduction of nucleic acid in cells. The present invention provides a method for introducing a polyanion into a cell or cells, and therefore can be used to introduce biologically active macromolecules or substances other than nucleic acids, including, among others, polyamines, polyamine acids, polypeptides, proteins, biotin, and polysaccharides into cells. Other useful materials for example, therapeutic agents, diagnostic materials and research reagents, can be introduced into cells by the methods of this invention. In a preferred aspect, any nucleic acid vector may be delivered to or into a cell by the present invention.

The methods of this invention can be used to generate transfected cells or tissues which express useful gene products. The methods of this invention can also be used as a step in the production of transgenic animals. The methods of this invention are useful in any therapeutic method requiring introducing of nucleic acids into cells or tissues. In particular, these methods are useful in cancer treatment, in in vivo and ex vivo gene therapy, and in diagnostic methods. See, for example, U.S. Pat. No. 5,589,466 to Felgner, et al. and U.S. patent application Ser. No. 08/450,555 filed on May 25, 1995 to Jessee, et al., herein incorporated by reference in their entireties. The transfection compounds or compositions of this invention can be employed as research reagents in any transfection of cells or tissues done for research purposes. Nucleic acids that can be transfected by the methods of this invention include DNA and RNA from any source comprising natural bases or non-natural bases, and include those encoding and capable of expressing therapeutic or otherwise useful proteins in cells or tissues, those which inhibit expression of nucleic acids in cells or tissues, those which inhibit enzymatic activity or activate enzymes, those which catalyze reactions (ribozymes), and those which function in diagnostic assays.

This invention also includes transfection kits which include one or more of the compounds or compositions of the present invention or mixtures thereof. Particularly, the invention provides a kit comprising one or more of the compounds of the present invention and at least one additional component selected from the group consisting of a cell, cells, a cell culture media, a nucleic acid, a transfection enhancer and instructions for transfecting a cell or cells.

### EXAMPLES

### Example 1

### Preparation and Testing of Self-Assembling Peptide Amphiphiles

Physical and chemical characterization. In experiments conducted during development of embodiments of the present invention, after HPLC purification, peptide amphiphiles (PA) were >95% pure by amide content. High resolution mass spectrometry confirmed identity. Gelation was observed both at 0.75 wt % at high pH for all PA systems and at 3.5 wt % with the addition of plasmid for the DNA-binding system. Circular dichroism and FT-IR both showed an α-helical signature for the DNA-binding system while the histidine hexamer showed a β-sheet signature. Physical morphology of the peptide amphiphile systems were characterized by transmission electron microscopy. In experiments conducted during development of embodiments of the present invention, it was found that the PA nanofibers changed morphology dramatically upon addition of plasmid. The DNA-binding PA alone was shorter, highly matted fibers whereas with DNA, the DNA-binding PA was highly bundled with long straight fibers.
Intact histidine hexamer PA is present inside cell aggregate. In experiments conducted during development of embodiments of the present invention, P19 cells in suspension were allowed to form aggregates for four days and were then cultured with the histidine hexamer PA, with sequence HHHHHHLLLAAA-palmitoyl for two weeks. Staining with anti-His₆ antibodies showed that the bioactive epitope was intact and bioavailable after two weeks in culture.
DNA binds to the DNA-binding PA. In experiments conducted during development of embodiments of the present invention, an electrophoretic mobility shift assay (EMSA) and a DNA footprinting experiment were conducted to demonstrate binding of the DNA binding PA to DNA. The EMSA showed that upon addition of the PA to the plasmid, the mobility of the plasmid was inhibited. There did not seem to be a preference for binding to the nicked, supercoiled, or nonsupercoiled forms. The DNA footprinting assay showed that while the PA did protect the DNA from degradation by DNase, there was no binding preference to a particular sequence of base pairs. DNase is an enzyme which binds to double-stranded DNA and degrades it. If the DNA is already bound, however, the DNA is protected from degradation by the DNase. Therefore, if less degradation is seen by gel electrophoresis, it is likely because the DNA has been bound by another ligand, preventing DNase from binding.
Transfection of aggregates is greatly enhanced. Experiments conducted during development of embodiments of the present invention showed that the PA system was nontoxic to P19 cells by flow cytometry at all tested concentrations. By contrast, lipofectamine is known to be toxic to cells at higher concentrations. P19 cells were allowed to form aggregates for seven days and then cultured with the DNA-PA complexes. The plasmid used here coded for GFP. A non-assembling peptide (the same bioactive epitope without the β-sheet forming region) and lipofectamine were used as controls. After two days, transfection efficiency was determined by qPCR and Western Blot. By the ΔΔCT method of PCR analysis, the PA system had an almost 10-fold increase in GFP mRNA transcripts in the largest aggregates (1.5 mm average diameter) and more than a seven-fold increase in smaller aggregates (0.4 mm average diameter). The Western Blot showed almost a three-fold increase in protein expressed for the largest aggregate size.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising one or more peptide amphiphiles, said peptide amphiphiles comprising a non-peptidic hydrophobic component covalently linked to structural peptide segment and a hydrophilic, DNA-binding peptide segment.

2. The composition of claim 1, wherein said structural peptide segment is configured to form a β-sheet.

3. The composition of claim 1, wherein said structural peptide segment is selected from any of SEQ ID NO. 5-16.

4. The composition of claim 1, wherein said DNA-binding peptide comprises RKTAKRLGVYQSAIN (SEQ ID NO 17).

5. The composition of claim 4, wherein said hydrophobic component is a single, linear alkyl chain of the formula: CₙH₂ₙ₋₁O—, where n=6−22.

6. The composition of claim 5 wherein the structural peptide segment is LLLAAA and the hydrophobic component is a palmitoyl moiety (C₁₆H₃₁O—).

7. The composition of claim 1, further comprising a peptide amphiphile comprising a non-peptidic hydrophobic component covalently linked to a structural peptide segment and a hydrophilic nuclear localization signaling segment.

8. The composition of claim 7, wherein said nuclear localization signal is a peptide comprising PPRKV (SEQ ID NO 19).

9. A composition comprising one or more peptide amphiphiles, said peptide amphiphiles comprising a non-peptidic hydrophobic component covalently linked to structural peptide segment and a hydrophilic, nuclear localization signaling segment.

10. The composition of claim 9, wherein said structural peptide segment is configured to form a β-sheet.

11. The composition of claim 9, wherein said structural segment is selected from any of SEQ ID NO. 5-16.

12. The composition of claim 9, wherein said nuclear localization signal is a peptide comprising PPRKV (SEQ ID NO 19).

13. The composition of claim 12, wherein said hydrophobic component is a single, linear alkyl chain of the formula: CₙH₂ₙ₋₁O—, where n=6−22.

14. The composition of claim 13, wherein the structural peptide segment is LLLAAA and the hydrophobic component is a palmitoyl moiety (C₁₆H₃₁O—).

15. The composition of claim 9, further comprising a composition of one or more peptide amphiphiles, said peptide amphiphiles comprising a non-peptidic hydrophobic component covalently linked to structural peptide segment and a hydrophilic, DNA-binding peptide segment.

16. A kit comprising the composition of claim 1.

17. A kit comprising the composition of claim 9.

18. A method of transfecting cells, comprising
a) providing: a plurality of cells, a peptide amphiphile composition, and a DNA target;
b) contacting said peptide amphiphile composition with said DNA target; and subsequently,
c) contacting said peptide amphiphile composition with said plurality of cells.

19. The method of claim 18, wherein said peptide amphiphile composition comprises one or more peptide amphiphiles, said peptide amphiphiles comprising a non-peptidic hydrophobic component covalently linked to structural peptide segment and a hydrophilic, DNA-binding peptide segment.

20. The method of claim 18, wherein said peptide amphiphile composition comprises one or more peptide amphiphiles, said peptide amphiphiles comprising a non-peptidic hydrophobic component covalently linked to structural peptide segment and a hydrophilic, nuclear localization signaling segment.

21. The method of claim 18, wherein said plurality of cells are a cellular aggregate.
